(19) United States
(12) Patent Application Publication
(10) Pub. No.: US 2024/0423221 A1
(43) Pub. Date: Dec. 26, 2024

(54) PANTOEA AGGLOMERANS STRAIN FL1 AND USE THEREOF IN IMPROVING DOUGH PROCESSING CHARACTERISTICS

(71) Applicant: Nanjing University Of Finance & Economics, Nanjing City (CN)

(72) Inventors: Guangyu Wang, Nanjing City (CN); Yi Zhang, Nanjing City (CN); Fang Ma, Nanjing City (CN); Fei Pei, Nanjing City (CN); Weifen Qiu, Nanjing City (CN)

(21) Appl. No.: 18/747,693

(22) Filed: Jun. 19, 2024

(30) Foreign Application Priority Data
Jun. 21, 2023 (CN) 202310744319.4

(51) Int. Cl. A21D 8/04

(52) U.S. Cl. CPC A21D 8/04

(57) ABSTRACT

Provided are a Pantoea agglomerans (P. agglomerans) strain FL1 and use thereof in improving dough processing characteristics, which belong to the technical field of food fermentation. The P. agglomerans strain FL1 was deposited on May 4, 2023, with a deposit number of CGMCC NO. 27248. Compared with that made using only common yeast, the dough made by co-fermentation of the P. agglomerans strain FL1 and yeast exhibits a more robust gluten protein network structure, increased resisillence during kneading, and reduced hardness, and results in bread with a larger volume and softer texture.

### CROSS REFERENCE TO RELATED APPLICATION

This patent application claims the benefit and priority of Chinese Patent Application No. 202310744319.4 filed with the China National Intellectual Property Administration on Jun. 21, 2023, the disclosure of which is incorporated by reference herein in its entirety as part of the present application.

### REFERENCE TO SEQUENCE LISTING

A computer readable XML file entitled “HLP20240402337_seqlist”, that was created on Apr. 26, 2024, with a file size of about 5,539 bytes, contains the sequence listing for this application, has been filed with this application, and is hereby incorporated by reference in its entirety.

### TECHNICAL FIELD

A Pantoea agglomerans strain FL1 and use thereof in improving dough processing characteristics are provided, belonging to the technical field of food fermentation.

### BACKGROUND

Bread, as a traditional staple food, is widely consumed in many countries around the world. With over 9 billion kilograms of bread products produced annually, global bread consumption averages about 70 kilograms per person per year. Consumers prefer bread with characteristics such as large volume, soft texture, uniform structure, and reasonable shelf life, which largely affect consumers' choice of the final product. In order to obtain a stable and robust network structure, desirable texture, and long shelf life in dough and bread, enzymes, lipids, hydrocolloids, emulsifiers, and some oxidants are generally added as additives and improvers in baked goods. However, as modern people incline to choose foods that are free of chemical preservatives and additives, it is more appropriate to replace chemical additives with microorganisms in the baking industry due to their higher safety and effectiveness.

Pantoea agglomerans is commonly found in a variety of environments including grains, soil, water, dust, dairy products, meat, fish, insects, human beings, and animals. Recent studies have found that P. agglomerans demonstrates unique metabolic capabilities, including the synthesis of many biologically-active substances. Moreover, P. agglomerans also shows the beneficial properties of biological control, prevention and treatment of animal diseases, and enhancement of body immune activity. However, there have been no reports on use of the P. agglomerans in improving dough and bread qualities so far.

### SUMMARY

An objective of the present disclosure is to provide a P. agglomerans strain FL1 and use thereof in improving dough processing characteristics, developing a new application field for P. agglomerans.

The present disclosure provides a P. agglomerans strain FL1 with a deposit number of CGMCC NO. 27248.

The present disclosure further provides use of the P. agglomerans strain FL1 in improving dough processing characteristics.

The present disclosure further provides use of the P. agglomerans strain FL1 combined with yeast in improving dough processing characteristics.

The present disclosure further provides a method for improving dough processing characteristics, including the following steps: mixing a bacterial suspension of the P. agglomerans strain FL1 with flour and yeast, and then conducting kneading and fermentation to produce a dough.

In some embodiments, the bacterial suspension of the P. agglomerans strain FL1 has a concentration of 10³ CFU/mL to 10⁸ CFU/mL.

In some embodiments, the bacterial suspension of the P. agglomerans strain FL1 is added at 45 to 60% of a mass of the flour.

In some embodiments, the fermentation is conducted at 29° C. to 31° C. for 80 min to 100 min.

In some embodiments, the flour includes whole wheat flour.

The present disclosure further provides a food product made from the dough prepared by the method.

In some embodiments, the food product includes bread.

### Beneficial Effects

The present disclosure provides a P. agglomerans strain FL1 and use thereof in improving dough processing characteristics. A dough fermented using the P. agglomerans strain FL1 has an increased proportion of α-helix and a more stable gluten protein network structure.

The P. agglomerans strain FL1 according to the present disclosure enhances the strength of the dough, providing a better stability at different frequencies and enabling the dough to withstand more intense kneading processes, resulting in a dough that is more resistant to kneading.

The P. agglomerans strain FL1 according to the present disclosure may affect the protein properties and water-holding capacity of flour, as well as the interaction between free water, flour lipids, and protein-starch, resulting in a dough with less hardness, and a bread that is softer, and larger in volume.

### Deposit of Biological Material

The P. agglomerans strain FL1 has been deposited in the China General Microbiological Culture Collection Center (CGMCC), No. 3, No. 1, West Beichen Road, Chaoyang District, Beijing on May 4, 2023, with a deposit number of CGMCC NO. 27248.

### BRIEF DESCRIPTION OF THE DRAWINGS

FIG. 1 shows a photo of the strain culture;

FIG. 2 shows an enlarged view of the strain culture;

FIG. 3 to FIG. 6 show scanning electron microscopy (SEM) images of the dough microstructure, where FIG. 3 shows the SEM image in Comparative Example 1, FIG. 4 shows that in Example 2, FIG. 5 shows that in Example 3, and FIG. 6 shows that in Example 4;

FIG. 7 shows the spectrum of a transparent sheet made from freeze-dried dough;

FIG. 8 shows moisture distribution of the doughs before fermentation;

FIG. 9 shows moisture distribution of the doughs after fermentation;

FIG. 10 shows the elastic moduli of the doughs;

FIG. 11 shows the viscous moduli of the doughs;

FIG. 12 shows the loss tangents of the doughs;

FIG. 13 shows the tensile strength and ductility of whole wheat doughs; and

FIG. 14 shows the spectrum of a transparent sheet made from a lyophilized whole wheat doughs.

### DETAILED DESCRIPTION OF THE EMBODIMENTS

The present disclosure provides a P. agglomerans strain FL1, which was deposited on May 4, 2023, with a deposit number of CGMCC NO. 27248.

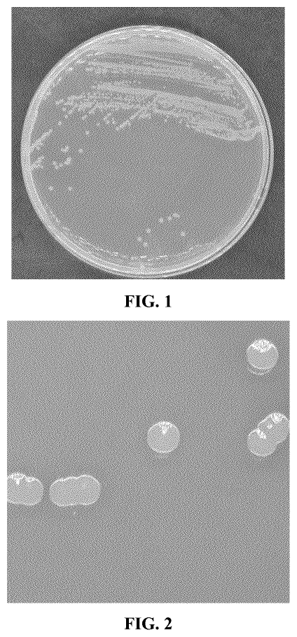

In the present disclosure, the P. agglomerans strain FL1 is Gram-staining negative. This strain can form 1.44 mm colonies after culturing on Luria-Bertani (LB) medium for 24 h. The colonies are light yellow and round with a convex surface, smooth and sticky, easy to lift, and have neat edges (FIG. 1 and FIG. 2).

In the present disclosure, the P. agglomerans strain FL1 may effectively improve the dough processing characteristics.

The present disclosure further provides use of the P. agglomerans strain FL1 in improving dough processing characteristics. The dough processing characteristics include: the gluten network structure of the fermented dough, hardness of the dough, content of protein secondary structure and moisture distribution in the dough, rheological attributes of the dough, and texture and specific volume of a prepared loaf of bread.

The present disclosure further provides use of the P. agglomerans strain FL1 combined with yeast in improving dough processing characteristics.

The P. agglomerans strain FL1 of the present disclosure is added when the dough is prepared. Compared with the dough made by fermentation with ordinary dry yeast alone, the dough using P. agglomerans strain FL1 has a more stable gluten protein network structure. The mechanisms of dough formation involve protein swelling, starch gelatinization, adhesion, and adsorption. These mechanisms involve interactions between various groups, hydrogen bonds, sulfur-hydrogen bonds, and disulfide bonds. Dough is also a foam system in which starch granules are wrapped in a network of gluten proteins. After fermentation by the P. agglomerans strain FL1, gluten proteins exist in a film-like form, forming a network structure, and the spherical starch granules wrapped in the gluten protein film play a role in supporting the network structure. The network structure becomes more stable when the structure is continuous and more starch granules are wrapped. Application of the P. agglomerans strain FL1 can increase the proportion of α-helix in the dough, thereby making the gluten protein network of the dough more stable.

In the present disclosure, the P. agglomerans strain FL1 is capable of promoting the complexation of gluten proteins in flour to form macromolecular aggregates, and enhancing the formation of intermolecular covalent bonds and hydrogen bonds. Due to the altered interactions between molecules, the characteristics of dough are affected, leading to stable and tight polypeptide chains. This improves the microstructure of the dough. In the examples, the rheological properties were measured. The P. agglomerans strain FL1 increased the elastic modulus (G′) and viscous modulus (G″) of the dough and promoted the complexation of gluten proteins in flour to form macromolecular aggregates. This, in turn, improves the strength of the dough, allowing it to exhibit better stability under different frequencies, and withstand more intense kneading processing, thus making the dough more resistant to kneading.

In the present disclosure, the P. agglomerans strain FL1 affects the protein properties and water-holding capacity of flour, as well as the interaction between free water, flour lipids, and protein-starch, resulting in a dough with less stiffness and bread with larger volume and softer texture.

The present disclosure further provides a method for improving dough processing characteristics, including the following steps: mixing a bacterial suspension of the P. agglomerans strain FL1 with flour and yeast, and conducting kneading and fermentation to produce a dough.

In the present disclosure, the P. agglomerans strain FL1 undergoes activation, first-stage expansion culture, and second-stage expansion culture, followed by dilution to prepare a bacterial suspension. The bacterial suspension of the P. agglomerans strain FL1 has a concentration of preferably 10³ CFU/mL to 10⁸ CFU/mL.

In the present disclosure, the bacterial suspension is mixed with a certain amount of flour and yeast for dough kneading. The bacterial suspension of the P. agglomerans strain FL1 is added at preferably 45% to 60%, more preferably 47% to 60% of a mass of the flour. The yeast is added at preferably 1% to 3%, more preferably 1% to 2% of the flour. In some embodiments, salt, sugar, and butter are also added when the dough is kneaded. The salt is added at preferably 1% to 2% of the flour, the sugar is added at preferably 5% to 6% of the flour, and the butter is added at preferably 3% to 5% of the flour.

In the present disclosure, the kneaded dough is fermented to prepare the dough. The fermentation is conducted at preferably 29° C. to 31° C., more preferably 30° C. to 31° C. for preferably 80 min to 100 min, more further preferably 85 min to 95 min, and most preferably 90 min. The fermentation is preferably conducted in a fermentation tank.

In the present disclosure, the flour preferably includes whole wheat flour. The whole wheat flour contains a large amount of bran, which results in a rough texture of the whole wheat dough, small bread volume, and increased bread crumb hardness, and makes the whole wheat products less acceptable. The P. agglomerans strain FL1 may effectively improve the quality of the whole wheat products.

The present disclosure further provides a food product made from the dough prepared by the method. The dough has a more stable gluten protein network structure, is more resistant to kneading, and shows lower stiffness. Moreover, the bread made from the dough exhibits a larger volume and a softer texture. In the examples, the specific volume of the bread was measured, and a significant increase in the specific volume of the bread further confirmed the improvement of dough characteristics by the P. agglomerans strain FL1.

In the present disclosure, the food product includes bread. The dough is baked in an oven to obtain bread.

In order to further illustrate the present disclosure, the P. agglomerans strain FL1 and the use thereof in improving dough processing characteristics provided by the present disclosure are described in detail below with reference to the accompanying drawings and examples, but the accompanying drawings and the examples should not be construed as limiting the protection scope of the present disclosure.

### Example 1

Ten grams of wheat flour were added to 90 mL of sterile physiological saline, thoroughly mixed by shaking, and then the sample was separately diluted 10 times and 100 times. From the above sample and its dilutions, 100 μL was plated separately on LB solid medium. The plates were incubated at 37° C. for 48 h. Putative P. agglomerans strains were randomly selected based on the morphological characteristics of colonies, and then purified by streaking on the LB solid medium. After incubation at 37° C. for 24 h, single colonies of the putative P. agglomerans strains were obtained. The putative strains were identified molecularly using 16SrDNA universal primers, including 27F (5′-AGAGTTTGATCCTGGCTCAG-3′, SEQ ID NO: 1) and 1492R (5′-CTACGGCTACCTTGTTACGA-3′, SEQ ID NO: 2). A small amount of target colonies were gently picked with a pipette tip and added to a prepared PCR reaction system. The polymerase chain reaction (PCR) was conducted, and the amplified PCR products were sequenced to obtain the 16SrDNA sequence of each putative P. agglomerans strain.

(1) BLAST search: the 16S rDNA sequences of each putative P. agglomerans strain were aligned in 16S ribosomal RNA sequences (Bacteria and Archaea) database of NCBI. It was found that the 16S rDNA sequence of strain FL1 had the highest homology with P. agglomerans strain NBRC 102470, with an identity of 99.03%. Combined with the morphological characteristics of the strain, the FL1 was identified as a member of P. agglomerans.

(2) The 16S rDNA sequence of the P. agglomerans strain FL1 was:

SEQ ID NO: 3
AGTGGTAAGCGCCCTCCCGAAGGTTAAGCTACCTACTTCTTTTGCAACC
CACTCCCATGGTGTGACGGGCGGTGTGTACAAGGCCCGGGAACGTATTC
ACCGTGGCATTCTGATCCACGATTACTAGCGATTCCGACTTCACGGAGT
CGAGTTGCAGACTCCGATCCGGACTACGACGCACTTTGTGAGGTCCGCT
TGCTCTCGCGAGGTCGCTTCTCTTTGTATGCGCCATTGTAGCACGTGTG
TAGCCCTACTCGTAAGGGCCATGATGACTTGACGTCATCCCCACCTTCC
TCCGGTTTATCACCGGCAGTCTCCTTTGAGTTCCCGACCGAATCGCTGG
CAACAAAGGATAAGGGTTGCGCTCGTTGCGGGACTTAACCCAACATTTC
ACAACACGAGCTGACGACAGCCATGCAGCACCTGTCTCACGGTTCCCGA
AGGCACTAAGGCATCTCTGCCGAATTCCGTGGATGTCAAGAGTAGGTAA
GGTTCTTCGCGTTGCATCGAATTAAACCACATGCTCCACCGCTTGTGCG
GGCCCCCGTCAATTCATTTGAGTTTTAACCTTGCGGCCGTACTCCCCAG
GCGGTCGACTTAACGCGTTAGCTCCGGAAGCCACTCCTCAAGGGAACAA
CCTCCAAGTCGACATCGTTTACGGCGTGGACTACCAGGGTATCTAATCC
TGTTTGCTCCCCACGCTTTCGCACCTGAGCGTCAGTCTTTGTCCAGGGG
GCCGCCTTCGCCACCGGTATTCCTCCAGATCTCTACGCATTTCACCGCT
ACACCTGGAATTCTACCCCCCTCTACAAGACTCAAGCCTGCCAGTTTCA
AATGCAGTTCCCAGGTTAAGCCCGGGGATTTCACATCTGACTTAACAGA
CCGCCTGCGTGCGCTTTACGCCCAGTAATTCCGATTAACGCTTGCACCC
TCCGTATTACCGCGGCTGCTGGCACGGAGTTAGCCGGTGCTTCTTCTGC
GGGTAACGTCAATCGGCGCGGTTATTAACCGCACCGCCTTCCTCCCCGC
TGAAAGTACTTTACAACCCGAAGGCCTTCTTCATACACGCGGCATGGCT
GCATCAGGCTTGCGCCCATTGTGCAATATTCCCCACTGCTGCCTCCCGT
AGGAGTCTGGACCGTGTCTCAGTTCCAGTGTGGCTGGTCATCCTCTCAG
ACCAGCTAGGGATCGTCGCCTAGGTGGGCCATTACCCCGCCTACTAGCT
AATCCCATCTGGGTTCATCCGATAGTGAGAGGCCCGAAGGTCCCCCTCT
TTGGTCTTGCGACGTTATGCGGTATTAGCCACCGTTTCCAGTGGTTATC
CCCCTCTATCGGGCAGATCCCCAGACATTACTCACCCGTCCGCCACTCG
TCACCCGAGAGCAAGCTCTCTGTGCTACCGTCCGACTTGCATGTGTTAG
GCGTGCGGCCAGCGTTCAATCTGAGC,.

(3) Genome-wide sequencing was conducted on the FL1 and the results were as followings:

The complete sequence obtained from the processing and assembly of the next- and third-generation sequencing data of strain FL1 was analyzed. It was found that the genome of FL1 consists of one chromosome and five plasmids.

The whole genome of strain FL1 was sequenced using Illumina NovaSeq combined with the PacBio Sequel sequencing platform. The statistics of the genome assembly data were shown in Table 1. Through the assembly and analysis of sequencing data, it was found that strain FL1 contains 1 chromosome and 5 plasmids, which are different from the genome assembly and annotation reports published in the NCBI database.

TABLE 1
Statistics of genome assembly data

| Sample | | | GC | |
|---|---|---|---|---|
| name | Sequence ID | Sequence length | content | Sequence type |
| FL1 | Chromosome | 4,017,180 | 55.28 | Circular genome |
| | Plasmid 1 | 504,646 | 53.51 | Circular genome |
| | Plasmid 2 | 161,161 | 52.36 | Circular genome |
| | Plasmid 3 | 45,770 | 50.43 | Circular genome |
| | Plasmid 4 | 40,075 | 50.73 | Circular genome |
| | Plasmid 5 | 3,341 | 44.12 | Circular genome |

Preparation of a Bacterial Suspension of the P. agglomerans Strain FL1.

First-stage activation: a standard solid LB medium was poured into a flat petri dish and cooled, the deposited P. agglomerans strain FL1 was inoculated, and then cultured at 37° C. for 48 h to obtain a solid medium after the first-stage expansion culture.

Second-stage expansion culture: a single colony was selected from the first-stage activation medium and transferred into 5 mL of a standard liquid LB medium, and cultured overnight at 37° C. with a 150 rpm shaker.

Third-stage expansion culture: the bacterial solution after the second-stage expansion culture was added to 5 mL of the liquid medium at an inoculum volume of 1%, and then cultured at 37° C. with a 150 rpm shaker for 8 h to obtain a bacterial suspension with a final concentration of 10⁸ CFU/mL. The abterial suspension was then diluted to different concentrations required for application, obtaining two bacterial suspensions with concentrations of 10⁶ CFU/mL and 10³ CFU/mL, respectively.

### Example 2

Preparation of doughs: 90 g of the bacterial suspension of the P. agglomerans strain FL1 with a final concentration of 10³ CFU/mL, 180 g of flour, 3 g of yeast, 2.5 g of salt, 10 g of sugar, and 5 g of butter were mixed to allow dough kneading, and the kneaded dough was placed in a fermentation tank to allow fermentation at 30° C. for 90 min to obtain a dough.

### Example 3

This example was conducted in a same manner as that in Example 2, except that the bacterial suspension of the P. agglomerans strain FL1 had a concentration of 10⁶ CFU/mL.

### Example 4

This example was conducted in a same manner as that in Example 2, except that the bacterial suspension of the P. agglomerans strain FL1 had a concentration of 10⁸ CFU/mL.

### Comparative Example 1

This comparative example was conducted in a same manner as that in Example 2, except that the bacterial suspension of the P. agglomerans strain FL1 was replaced with water.

### Example 5

This example adopted an alternative culture process of the P. agglomerans strain FL1 in Example 1:

After activation on LB solid medium, a single colony was picked and transferred to 5 mL liquid medium allow expansion culture for 12 h. Then a fixed proportion (1%) of the culture was transferred to 40 mL of LB liquid medium to allow secondary expansion culture. According to the growth curve of FL1, the culture time was determined to be 8 h (the bacteria were in the logarithmic growth phase at this time) and a concentration of the bacterial solution at this time was 10⁸ CFU/mL after counting the colonies.

### Example 6

Test method for the influence on a microstructure of the dough: the prepared dough was immediately frozen in a refrigerator at −20° C. overnight and then lyphilized. The lyphilized dough was ground into particles to measure the microstructure. A cross-section of the lyphilized dough was sprayed with gold and placed on the stage of SEM (TM3000) for observation. Images were acquired with a magnification of 2.0 k× at an accelerating voltage of 5 kV.

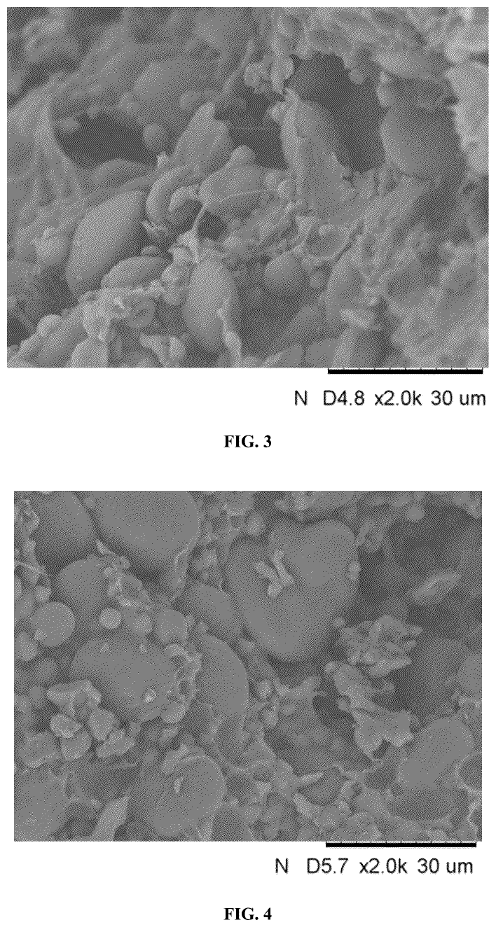

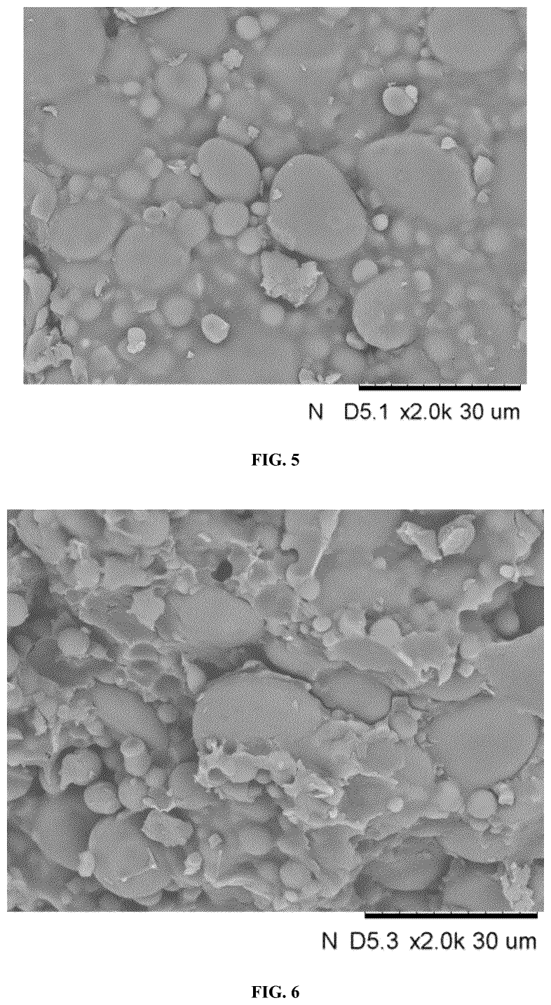

As shown in FIG. 3 to FIG. 6, the dough in Comparative Example 1 showed a discontinuous and disordered structure with multiple voids and ditches, and the starch granules were not completely retained in the gluten protein network, indicating that the gluten protein network was weak. The dough of Examples 2 to 4 (FIG. 4 to FIG. 6) formed a continuous and compact structure, with more small starch granules appearing and being wrapped in the gluten matrix, indicating that the P. agglomerans strain FL1 strengthened the gluten network of dough and made it more stable.

### Example 7

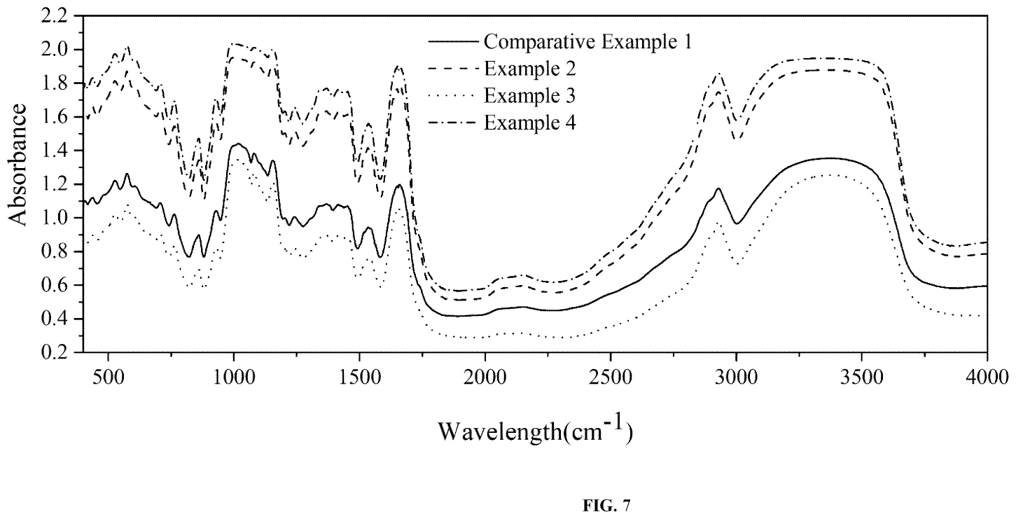

Test method for the influence on the secondary structure of protein in dough: a transparent sheet was prepared by grinding the lyophilized dough into powder and mixing it with potassium bromide in a mortar at a mass ratio of 1:100. The spectra of the transparent sheet were measured by an FTIR spectrometer. Against the background of a scanning frequency of 64, the absorbance of infrared radiation was recorded at 4,000 cm⁻¹ to 400 cm⁻¹ with an interval of 4 cm⁻¹. The spectral analysis of each sample was conducted using PeakFit 4.12 software to obtain the spectra (FIG. 7), and the total secondary structure content in the obtained dough is shown in Table 2.

TABLE 2
Total secondary structure content in dough

| | Total secondary structure content (%) | | | |
|---|---|---|---|---|
| Sample | α-helix | β-sheet | β-turn | Random coil |
| Comparative | 11.78% ± 0.002c | 26.48% ± 0.001a | 38.12% ± 0.001a | 23.62% ± 0.004b |
| Example 1 | | | | |
| Example 2 | 14.91% ± 0.003b | 26.01% ± 0.002b | 33.01% ± 0.002b | 26.07 ± 0.002a |
| Example 3 | 27.04% ± 0.005a | 25.92% ± 0.02b | 34.46% ± 0.004b | 12.57% ± 0.003c |
| Example 4 | 14.72% ± 0.004b | 26.58% ± 0.004a | 35.20% ± 0.002b | 23.50% ± 0.001b |

As shown in Table 2, compared with Comparative Example 1, the proportion of α-helix increased in the total secondary structure in dough of Examples 2 to 4. The α-helix conformation has higher hydrophobicity and rigidity other structures. The higher the proportion of the α-helix, the higher the hydrophobicity. Therefore, the increase in the proportion of α-helix conformation indicated that the addition of P. agglomerans strain FL1 made the protein network structure of the dough more stable.

### Example 8

Test method for moisture distribution in dough: the moisture distribution of dough before and after fermentation was separately measured with a nuclear magnetic resonance imaging (NMRI) analyzer to study the water retention capacity of the dough. Experiments were conducted after dough preparation, referring to the literature: Sun L, Li X, Zhang Y, et al. A novel lactic acid bacterium for improving the quality and shelf life of whole wheat bread [J]. Food Control, 2020, 109:106914.

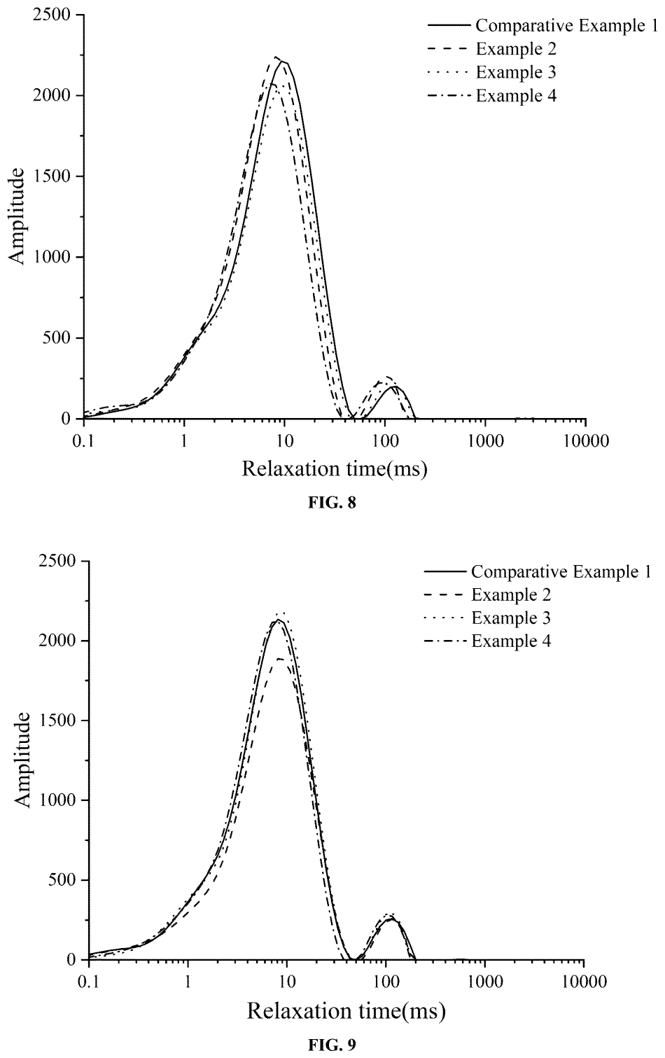

FIG. 8 and FIG. 9 represent the moisture distribution in the dough before and after fermentation, respectively. It could be seen that both before and after fermentation, the moisture distribution in the dough mainly had two parts, namely T₂₂ and T₂₃, which were weakly bound water and free water, with the weakly bound water being dominant.

TABLE 3
Moisture content in dough before and after fermentation

| | T₂₂/ms | | T₂₃/ms | | A₂₂/% | | A₂₃/% | |
|---|---|---|---|---|---|---|---|---|
| | Before | After | Before | After | Before | After | Before | After |
| | fermen- | fermen- | fermen- | fermen- | fermen- | fermen- | fermen- | fermen- |
| | tation | tation | tation | tation | tation | tation | tation | tation |
| Comparative | 9.33 | 8.11 | 132.19 | 114.98 | 97.04 | 95.58 | 2.96 | 4.42 |
| Example 1 | | | | | | | | |
| Example 2 | 8.11 | 8.11 | 100.00 | 114.98 | 96.39 | 95.73 | 3.61 | 4.27 |
| Example 3 | 9.33 | 8.11 | 114.98 | 114.98 | 96.46 | 95.50 | 3.54 | 4.50 |
| Example 4 | 7.05 | 8.11 | 100.00 | 100.00 | 95.95 | 95.33 | 3.99 | 4.67 |

As shown in Table 3, the T₂₂ and T₂₃ of the doughs in Examples 2 to 4 before fermentation were reduced, with those in Example 2 and Example 4 being more significant. This indicates that the addition of the FL1 strain caused a reduction in the degree of freedom of water in the dough, leading to a tighter binding with other components. The T₂₂ and T₂₃ of the dough after fermentation remained relatively unchanged. After 1.5 h of fermentation, the gluten network structure of the dough underwent similar disruptions, resulting in a similar state. In addition, before the dough was fermented, the addition of P. agglomerans strain FL1 resulted in a decrease in the percentage of weakly bound water and an increase in the content of free water, indicating a reduced water-holding capacity of the dough and a redistribution of water. After fermentation, the moisture content of each component of the dough remained basically unchanged.

### Example 9

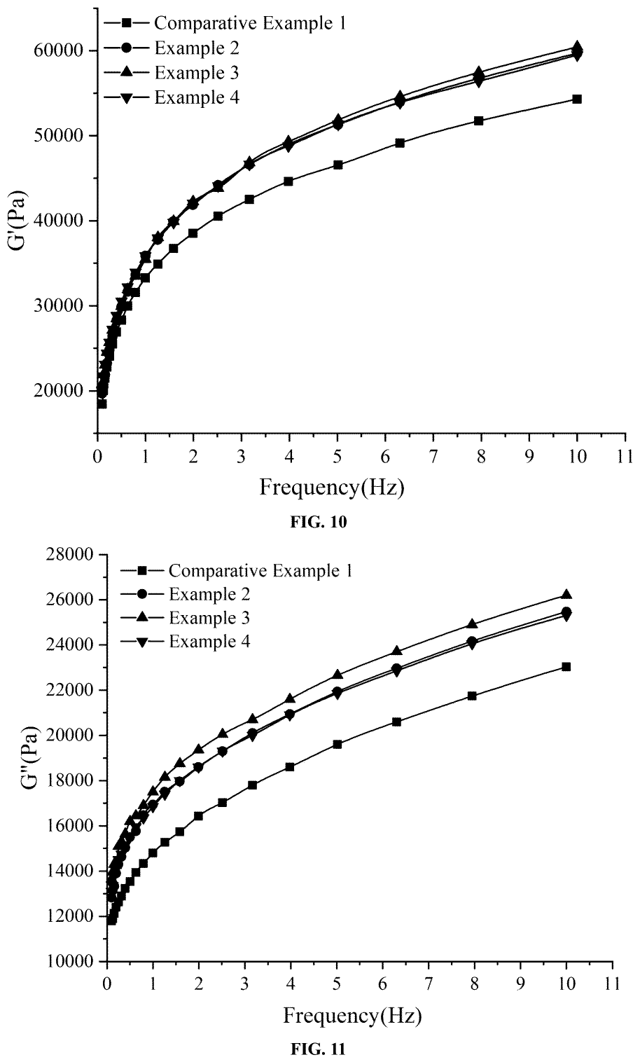

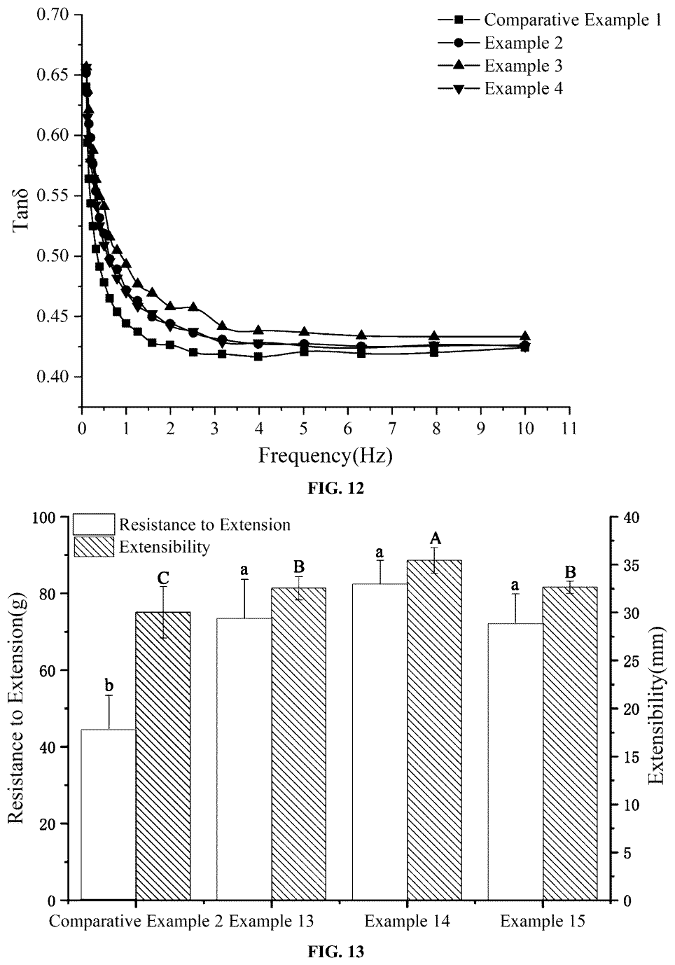

Test method for the influence on the rheological properties of dough: immediately after preparation, the dough was placed in the center of a rheometer rack for 5 min to release residual stress. The edges of dough were coated with mineral oil to prevent water loss. A PP25 rotor with a flat plate diameter of 25 mm was used, and the distance between the flat plates was set to 1 mm. During the test, a stress of 0.1% was applied at a test temperature of 25±1° C. and a test frequency of 0.1 Hz to 10 Hz to obtain the dough's elastic modulus (G′) (FIG. 10), viscous modulus (G″) (FIG. 11), and loss tangent value (i.e., tan δ=G″/G′, FIG. 12) as they change with frequency.

It can be seen from the above figure that the G′ of all doughs is significantly higher than G″. The G′ and G″ of the doughs in Examples 2 to 4 increased as the frequency increased. This indicates that the doughs have solid-like properties and the addition of P. agglomerans strain FL1 promotes the complexation of gluten proteins in flour to form macromolecular aggregates, as well as promotes covalent bonding and hydrogen bonding between molecules, thereby forming stable and tight polypeptide chains, enhancing the strength of the dough. In addition, the dough of Example 4 had the best viscoelasticity. Changes in the loss tangent value reflect changes in the number of polymers and the degree of polymerization in the dough system. The smaller this value is, the greater the elasticity ratio of the dough and the greater the number of polymers are. In summary, it is proved that the addition of P. agglomerans strain FL1 enables the dough to have better stability under different frequencies, allowing it to withstand more intense kneading and processing.

### Example 10

Preparation of bread: the dough samples prepared in Examples 2 to 4 were baked in an oven at 180° C. for 15 min to obtain bread.

### Example 11

Test method for the impact on bread texture: the texture of bread was measured using a food physical instrument. Measurement conditions included: P/36R probe, pre-test speed 3 mm/s, test speed 1 mm/s, post-test speed 3 mm/s, trigger automatic 10 g, measurement ratio 50%, interval 5 s. The baked bread was cooled to room temperature and then sliced. Then six samples were stacked with every two on top of each other, that is, two small slices of bread from the same treatment group were stacked together, and each sample group was measured 6 times.

TABLE 4
Impact on bread texture

| | Comparative | | | |
|---|---|---|---|---|
| | Example 1 | Example 2 | Example 3 | Example 4 |
| Hardness | 39.893 ± 6.87a | 34.017 ± 4.48ab | 24.878 ± 9.41c | 26.042 ± 1.10bc |
| Brittleness | 27.3558 ± 10.62a | 33.536 ± 3.67b | 11.034 ± 2.14c | 25.7535 ± 0.95a |
| Elasticity | 0.841 ± 0.04a | 0.845 ± 0.01a | 0.827 ± 0.02a | 0.829 ± 0.01a |
| Cohesiveness | 0.689 ± 0.05c | 0.756 ± 0.04ab | 0.686 ± 0.11c | 0.794 ± 0.02a |
| Gumminess | 27.5 ± 5.08a | 25.56 ± 2.20a | 16.407 ± 3.88b | 20.694 ± 1.08b |
| Chewiness | 23.103 ± 4.18a | 21.592 ± 2.09a | 13.516 ± 3.02c | 17.166 ± 1.16b |
| Resilience | 0.225 ± 0.04c | 0.29 ± 0.03a | 0.273 ± 0.08ab | 0.329 ± 0.01a |

As shown in Table 4, the hardness of the bread in Examples 2 to 4 was significantly reduced, indicating that the P. agglomerans strain FL1 inhibits the aging of the bread. Elasticity refers to the degree to which a sample returns to its original shape after the first compression, and is positively related to the quality of bread; the higher the elasticity is, the softer the bread can be. The use of the P. agglomerans strain FL1 had almost no influence on the elasticity of the bread, but reduced the chewiness.

### Example 12

Test method for the influence on the specific volume of bread: the cooled bread was accurately weighed, the volume was measured using the rice discharge method, and three parallel experiments were conducted. The specific volume (mL/g) was calculated based on the volume-to-mass ratio of bread.

TABLE 5
Influence on specific volume for bread
(analytical error and significance)

| Sample | Specific volume (cm³/g) |
|---|---|
| Comparative Example 1 | 3.01 ± 0.03c |
| Example 2 | 3.20 ± 0.03b |
| Example 3 | 3.43 ± 0.05a |
| Example 4 | 3.24 ± 0.04b |

As shown in Table 5, the specific volume of the bread of Examples 2 to 4 is significantly higher than that of Comparative Example 1. It can seen that the P. agglomerans strain FL1 increased the specific volume of the dough, indicating that the bread became fluffier and is more likely to be liked by consumers.

### Example 13

This example was conducted in the same manner as that in Example 2, except that the flour was replaced with whole wheat flour, and the bacterial suspension of P. agglomerans strain FL1 with a concentration of 10⁻³ CFU/mL was added at 60% of a mass of the whole wheat flour.

### Example 14

This example was conducted in the same manner as that in Example 3, except that the flour was replaced with whole wheat flour, and the bacterial suspension of P. agglomerans strain FL1 with a concentration of 10⁶ CFU/mL was added at 60% of a mass of the whole wheat flour.

### Example 15

This example was conducted in the same manner as that in Example 4, except that the flour was replaced with whole wheat flour, and the bacterial suspension of P. agglomerans strain FL1 with a concentration of 10⁸ CFU/mL was added at 60% of a mass of the whole wheat flour.

### Comparative Example 2

This comparative example was conducted in the same manner as that in Comparative Example 1, except that the flour was replaced with whole wheat flour.

### Example 16

Test method for the influence on tensile properties of whole wheat dough: the influence of different concentrations of FL1 on the tensile properties of whole wheat dough was measured with a TA-XTPlus texture analyzer (Stable Micro Systems, London, UK). The probe was an A/KIE probe, and the pre-test, test, and post-test speeds were 2 mm/s, 2 mm/s, and 10 mm/s, respectively. The triggering force was 5 g and the measuring distance was 30 mm. Each set of samples was repeatedly tested six times.

As the two most important parameters in the tensile curve, tensile strength and ductility can reflect the fluidity and tensile strength of the dough. As shown in FIG. 13, different concentrations of FL1 significantly improved the tensile strength and ductility of whole wheat dough, and the improvement was more significant when the concentration of FL1 was 10⁶ CFU/mL. This indicates that the addition of FL1 enables the gluten network structure of whole wheat dough to be formed more fully and enhances the strength of the dough.

### Example 17

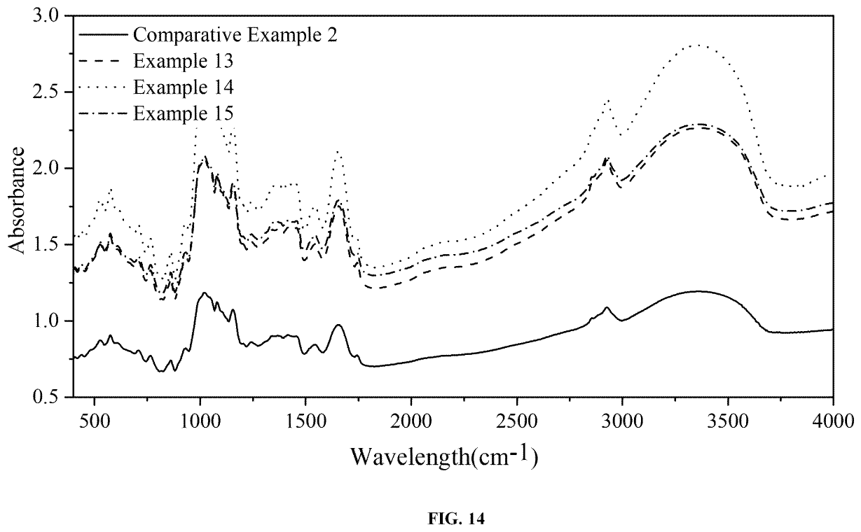

Test method for the impact on protein secondary structure in whole wheat dough: the spectra were detected using the method of Example 7 (FIG. 14), and the total secondary structure content in the obtained dough was detailed in Table 6.

As shown in FIG. 14, the dough in Examples 13 to 15 had higher Fourier transformation infrared absorption than that in Comparative example 12. As shown in Table 6, compared with Comparative Example 2, the proportions of β-sheets, random coils, and α-helix increased and that of β-turns decreased in the total secondary structure in the dough of Examples 13 to 15. It may be attributed to the transformation of β-turns to other conformations during the fermentation. The β-sheet conformation is the most stable secondary structure in gluten protein and the α-helix has higher hydrophobicity and rigidity compared to other confirmations. Therefore, the addition of P. agglomerans strain FL1 makes the protein network structure of the whole wheat dough more stable.

TABLE 6
Total secondary structure content in whole wheat dough

| | Total secondary structure content (%) | | | |
|---|---|---|---|---|
| Sample | α-helix | β-sheet | β-turn | Random coil |
| Comparative | 15.69% ± 0.004c | 43.57% ± 0.003b | 27.48% ± 0.008b | 13.26% ± 0.009b |
| Example 2 | | | | |
| Example 13 | 17.66% ± 0.001a | 32.80% ± 0.001a | 32.94% ± 0.003a | 16.60% ± 0.002a |
| Example 14 | 17.77% ± 0.002a | 33.49% ± 0.001a | 32.74% ± 0.002a | 16.00% ± 0.003a |
| Example 15 | 16.23%0.01b | 34.08% ± 0.01a | 33.40% ± 0.005a | 16.29% ± 0.005a |

### Example 18

Test method for the impact on the texture of whole wheat bread: the method of Example 11 was adopted to detect the impact on the texture of whole wheat bread, and the impact on the texture of whole wheat bread are shown in Table 7.

TABLE 7
Influence on texture in whole wheat bread

| | Comparative | | | |
|---|---|---|---|---|
| | Example 2 | Example 13 | Example 14 | Example 15 |
| Hardness | 2754.48 ± 19.47a | 2643.96 ± 32.30b | 2448.03 ± 30.43c | 2644.75 ± 29.19b |
| Elasticity | 0.83 ± 0.08a | 0.69 ± 0.08b | 0.83 ± 0.09a | 0.8 ± 0.05a |
| Cohesiveness | 0.72 ± 0.07a | 0.69 ± 0.08a | 0.73 ± 0.06a | 0.66 ± 0.08a |
| Gumminess | 1665.12 ± 18.12c | 1712.57 ± 11.22b | 1766.3 ± 16.06b | 1817.53 ± 22.29a |
| Chewiness | 1414.16 ± 18.68a | 1240.98 ± 23.17c | 1351.81 ± 30.48b | 1338.61 ± 30.27b |
| Resilience | 0.29 ± 0.03a | 0.25 ± 0.04b | 0.27 ± 0.02ab | 0.24 ± 0.03b |

As shown in Table 7, different concentrations of FL1 reduced the hardness, elasticity, chewiness, and resilience of whole wheat bread, and increased the gumminess. The cohesiveness did not change significantly compared with that of Comparative Example 2. This is because hardness, cohesiveness, and chewiness are negatively correlated with bread quality, while gumminess is positively correlated with bread quality.

Therefore, considering the changes in various indicators, the presence of FL1 will reduce the unique high hardness characteristics of whole wheat bread and improve the quality of the bread to a certain extent.

Although the above example has described the present disclosure in detail, it is only a part of, not all of, the embodiments of the present disclosure. Other embodiments may also be obtained by persons based on the example without creative efforts, and all of these embodiments shall fall within the protection scope of the present disclosure.

What is claimed is:

1. A method for improving dough processing characteristics, comprising the following steps: mixing a bacterial suspension of a Pantoea agglomerans (P. agglomerans) strain FL1 with flour and yeast, and conducting kneading and fermentation to obtain a dough, wherein the P. agglomerans strain FL1 has a deposit number of CGMCC NO: 7248.

2. The method according to claim 1, wherein the bacterial suspension of the P. agglomerans strain FL1 has a concentration of 10³ colony forming unit (CFU)/mL to 10⁸ CFU/mL.

3. The method according to claim 1, wherein the bacterial suspension of the P. agglomerans strain FL1 is added at 45% to 60% of a mass of the flour.

4. The method according to claim 1, wherein the fermentation is conducted at 29° C. to 31° C. for 80 min to 100 min.

5. The method according to claim 1, wherein the flour comprises whole wheat flour.

6. A food product made from the dough prepared by the method according to claim 1.

7. The food product according to claim 6, comprising bread.

8. The food product according to claim 6, wherein the bacterial suspension of the P. agglomerans strain FL1 has a concentration of 10³ CFU/mL to 10⁸ CFU/mL.

9. The food product according to claim 6, wherein the bacterial suspension of the P. agglomerans strain FL1 is added at 45% to 60% of a mass of the flour.

10. The food product according to claim 6, wherein the fermentation is conducted at 29° C. to 31° C. for 80 min to 100 min.

11. The food product according to claim 6, wherein the flour comprises whole wheat flour.


SEQUENCE LISTING (3 sequences)

SEQ ID NO: 1 - DNA, 20 bp, synthetic construct
  source 1..20: mol_type = other DNA; note = Primer 27F
agagtttgatcctggctcag

SEQ ID NO: 2 - DNA, 20 bp, synthetic construct
  source 1..20: mol_type = other DNA; note = Primer 1492R
ctacggctaccttgttacga

SEQ ID NO: 3 - DNA, 1447 bp, Pantoea agglomerans
  source 1..1447: mol_type = genomic DNA; note = 16S rDNA of Pantoea agglomerans strain FL1
agtggtaagcgccctcccgaaggttaagctacctacttcttttgcaacccactcccatggtgtgacgggcggtgtgtacaaggcccgggaacgtattcaccgtggcattctgatccacgattactagcgattccgacttcacggagtcgagttgcagactccgatccggactacgacgcactttgtgaggtccgcttgctctcgcgaggtcgcttctctttgtatgcgccattgtagcacgtgtgtagccctactcgtaagggccatgatgacttgacgtcatccccaccttcctccggtttatcaccggcagtctcctttgagttcccgaccgaatcgctggcaacaaaggataagggttgcgctcgttgcgggacttaacccaacatttcacaacacgagctgacgacagccatgcagcacctgtctcacggttcccgaaggcactaaggcatctctgccgaattccgtggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccactcctcaagggaacaacctccaagtcgacatcgtttacggcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactcaagcctgccagtttcaaatgcagttcccaggttaagcccggggatttcacatctgacttaacagaccgcctgcgtgcgctttacgcccagtaattccgattaacgcttgcaccctccgtattaccgcggctgctggcacggagttagccggtgcttcttctgcgggtaacgtcaatcggcgcggttattaaccgcaccgccttcctccccgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgggccattaccccgcctactagctaatcccatctgggttcatccgatagtgagaggcccgaaggtccccctctttggtcttgcgacgttatgcggtattagccaccgtttccagtggttatccccctctatcgggcagatccccagacattactcacccgtccgccactcgtcacccgagagcaagctctctgtgctaccgtccgacttgcatgtgttaggcgtgcggccagcgttcaatctgagc